(19) United States
(12) Patent Application Publication
(10) Pub. No.: US 2020/0246570 A1
(43) Pub. Date: Aug. 6, 2020

(54) NON-REBREATHER FACE MASK

(71) Applicants: Chris Salvino, Scottsdale, AZ (US); Keir Hart, Lafayette, CO (US)

(72) Inventors: Chris Salvino, Scottsdale, AZ (US); Keir Hart, Lafayette, CO (US)

(21) Appl. No.: 16/857,150

(22) Filed: Apr. 23, 2020

Related U.S. Application Data
(63) Continuation-in-part of application No. 15/394,704, filed on Dec. 29, 2016.
Application No. 15/394,704 is a continuation of application No. 15/122,119, filed on Aug. 26, 2016, now abandoned.
Application No. 15/122,119 is a continuation of application No. PCT/US2015/018021, filed on Feb. 27, 2015.
(60) Provisional application No. 61/946,602, filed on Feb. 28, 2014.

(51) Int. Cl. A61M 16/06; A61M 16/00; A61M 16/20; A61M 16/08

(52) U.S. Cl. CPC A61M 16/0605; A61M 16/0078; A61M 16/0875; A61M 16/06; A61M 16/0816; A61M 16/208

(57) ABSTRACT

Disclosed herein are embodiments describing nonrebreather facemasks for efficiently and comfortably delivering oxygen to patients. One embodiment describes a cup-shaped pliable facemask that is suited to cover and seal a patient's nose, mouth, and cheeks within the cup-shaped facemask. Certain other embodiments describe an inlet tube outwardly extending from the facemask at an angle in line with the pathway of a patient's nostrils to better provide oxygen directly into a patient's nose. Other embodiments envision varying facemask's stiffness for improved comfort and sealing against the patient's face. While other embodiments envision a reduction in dead space of a facemask when worn by a patient to improve oxygen efficiency used by the patient.

### CROSS-REFERENCE TO RELATED APPLICATIONS

This application claims priority to U.S. patent application Ser. No. 15/394,704 filed Dec. 29, 2016 entitled NON-REBREATHER FACE MASK, the contents of which are hereby incorporated by reference, U.S. patent application Ser. No. 15/394,704 which claims priority to and the benefit of U.S. patent application Ser. No. 15/122,119 entitled NON-REBREATHER FACE MASK filed Aug. 26, 2016, PCT application PCT/2015US/018021 filed Feb. 27, 2015, and to U.S. provisional application No. 61/946,602 filed Feb. 28, 2014, the contents of which are hereby incorporated by reference.

### FIELD OF THE INVENTION

The present embodiments are directed to nonrebreather facemasks for efficiently delivering oxygen to patients.

### DESCRIPTION OF RELATED ART

Non-rebreather face (NRB) and regular face masks (RFM) are used in medical emergencies where people in distress require oxygen therapy. Such emergencies include physical trauma, chronic airway limitations, chronic obstructive pulmonary diseases, smoke inhalation, carbon monoxide poisoning and other respiratory impairments.

A typical NRB facemask covers both the nose and the mouth of a patient. It is constrained to their face by way of an elastic strap that goes around the back of the patient's head. The elastic strap is essentially attached to the perimeter of the typical facemask (within a quarter/half an inch from the perimeter). Facemasks used today are somewhat inefficient and uncomfortable. It is to innovations related to this subject matter that the embodiments of the invention is generally directed.

### SUMMARY OF THE INVENTION

The present embodiments are directed to facemasks, such as nonrebreather facemasks, for efficiently and comfortably delivering oxygen to patients.

Certain embodiments of the present invention contemplate a variable stiffness facemask comprising: a cup shaped pliable facemask that is essentially defined by a nose covering region, a mouth covering region, two cheek coving regions and a perimeter, the perimeter essentially defined by a nose bridge rim, two cheek rims and a chin rim, the nose covered region shaped to closely conform to a human nose, the mouth covering region configured to be in front of a human mouth, the cheek covering regions configured to at least partially contact the corresponding sides of each human cheek; an inlet tube outwardly extending from the facemask, the inlet tube generally defined by a passageway that leads through a distal aperture and a nose aperture in the facemask; at least one strap anchor between the inlet tube and the mouth covering region; and a low force deflection band that extends at least 0.3 inches from the perimeter towards the mouth covering region, the low force deflection band being at least 15% more pliable than the mouth covering region.

Other certain embodiments of the present invention contemplate a nasal directed flow facemask comprising: a pliable cup shaped facemask that is essentially defined by a nose covering region, a mouth covering region, two cheek coving regions and a perimeter, the perimeter essentially defined by a nose bridge rim, two cheek rims and a chin rim, the nose covered region shaped to closely conform to a human nose, the mouth covering region configured to be in front of a human mouth, the cheek covering regions configured to at least partially contact the corresponding sides of each human cheek; an inlet tube outwardly extending from the facemask, the inlet tube generally defined by a passageway that leads through a distal aperture and nose aperture in the facemask, the inlet tube extending in a downward direction from the facemask at an angle between 30° and 60° from horizontal when the facemask is worn on a human face and the human face is in a neutral position.

Yet other certain embodiments of the present invention contemplate a nasal directed flow facemask comprising: a pliable cup shaped facemask that is essentially defined by a nose covering region, a mouth covering region, two cheek coving regions and a perimeter, the perimeter essentially defined by a nose bridge rim, two cheek rims and a chin rim, the nose covered region shaped to closely conform to a human nose, the mouth covering region configured to be in front of a human mouth, the cheek covering regions configured to at least partially contact the corresponding sides of each human cheek; an inlet tube outwardly extending from the facemask, the inlet tube generally defined by a passageway that leads through distal aperture and nose aperture in the facemask, a facemask cup volume that is essentially defined by a maximum amount of water the facemask can hold, the facemask cup volume less than 80 cc (5 in³).

### BRIEF DESCRIPTION OF THE DRAWINGS

FIG. 1 is a line drawing of a person wearing a nasal directed flow nonrebreather facemask, or simply facemask, consistent with embodiments of the present invention;

FIG. 2A is an isometric line drawing of the facemask embodiment consistent with embodiments of the present invention;

FIG. 2B illustratively depicts a side view line drawing of a facemask embodiment showing one of two touch point flares consistent with embodiments of the present invention;

FIG. 2C is a side view line drawing of just the facemask cup portion of a facemask embodiment consistent with embodiments of the present invention;

FIG. 2D illustratively depicts the location of the different regions in a facemask embodiment consistent with embodiments of the present invention;

FIGS. 3A and 3B are isometric line drawings of a facemask embodiment with various head strap arrangements consistent with embodiments of the present invention;

FIGS. 4A and 4B illustratively depict line drawings of interior volume and dead space of a facemask embodiment consistent with embodiments of the present invention;

FIGS. 5A-5C are side view line drawings of a variable stiffness facemask embodiment consistent with embodiments of the present invention;

FIGS. 6A-6E illustratively depict various diffuser embodiments located in the inlet tube passageway of a facemask embodiment consistent with embodiments of the present invention;

and

FIG. 7 is a line drawing of yet a different embodiment of a filter facemask without an air inlet port and a filter over the mouth region consistent with embodiments of the present invention.

### DETAILED DESCRIPTION

Initially, this disclosure is by way of example only, not by limitation. Thus, although the instrumentalities described herein are for the convenience of explanation, shown and described with respect to exemplary embodiments, it will be appreciated that the principles herein may be applied equally in other types of instruments and situations involving aspects of the inventive concepts of the disclosed nonrebreather facemasks. In what follows, similar or identical structures may (and may not) be identified using identical callouts.

Disclosed herein are embodiments describing nonrebreather facemasks for efficiently and comfortably delivering oxygen to patients. One embodiment describes a cup-shaped pliable facemask that is suited to cover and seal a patient's nose, mouth, and cheeks within the cup-shaped facemask. Certain other embodiments describe an inlet tube outwardly extending from the facemask at an angle in line with the pathway of a patient's nostrils to better provide oxygen directly into a patient's nose. Other embodiments envision varying facemasks stiffness for improved comfort, sealing against the patient's face and improved fitting for different face geometries and sizes. While other embodiments envision a reduction in dead space of a facemask when worn by a patient to improve oxygen efficiency used by the patient. Some embodiments envision a reduction in dead space as described below can improve oxygen purity levels breathed in by a patient to be greater than 80% due of a reduction in CO₂ exhalent from the patient that normally sits in the dead space in the facemask while being worn. Yet additional embodiments envision placement of a retaining strap across the facemask or optionally anchored just below the nose portion on either side of the nose portion of the facemask to reduce dead space and provide flexibility to reduce the potential for obstructing a person's nostrils base on a variety of different face shapes and sizes. Still other embodiments envision a diffuser in the inlet to break up or otherwise change incoming laminar oxygen flow to turbulent oxygen flow to improve comfort of oxygen flowing in a patient's nostrils. These embodiments of the present invention more generally relate to a detailed description in view of the associated figures illustrating examples of the inventive concepts presented below.

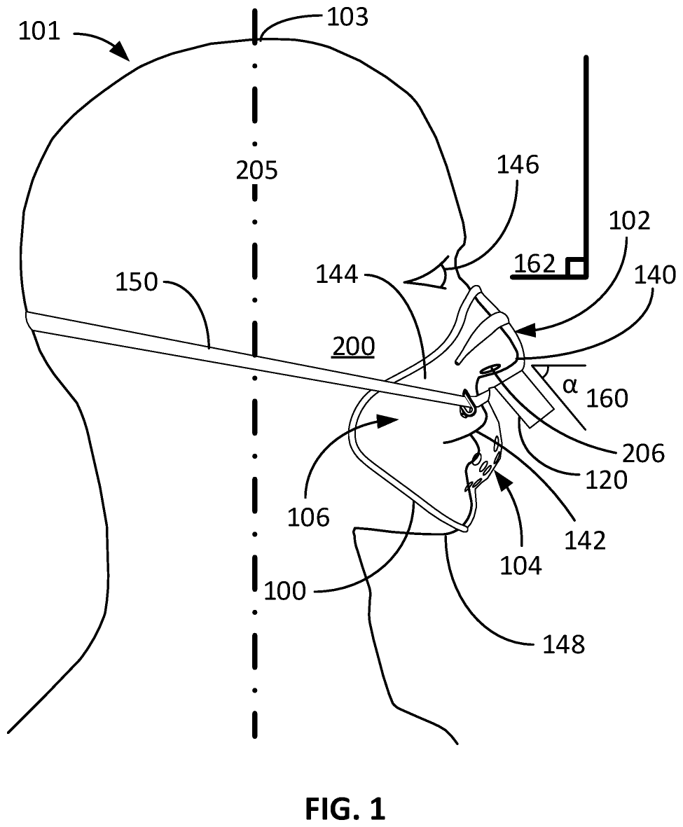

FIG. 1 is a line drawing of a person wearing a nasal directed flow nonrebreather facemask, or simply facemask, consistent with embodiments of the present invention. The person's head 101 is in a neutral position 205 whereby their eyes 146 are neutrally looking forward along the horizontal plane 162 with the crown 103 of their head 101 along the neutral axis 205 essentially at a right angle to the horizontal plane 162. In this neutral position 205, the nose inlet tube 120 is outwardly extending from the facemask 100 at an angle (a) 160 between 30° and 60° and preferably between 42° and 50°. Some embodiments envision the angle (a) 160 at approximately 48°. Airflow is directed at the angle (a) 160 into the person's nostrils 206 at the intake angle 160 to improve oxygen flow to the person's nostrils 206 to increase oxygen uptake by the person/patient 101.

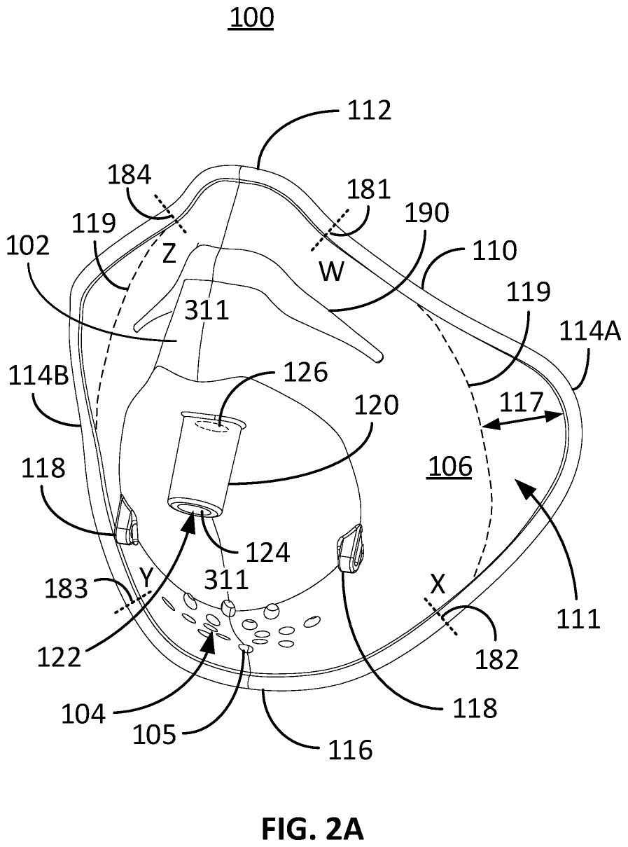

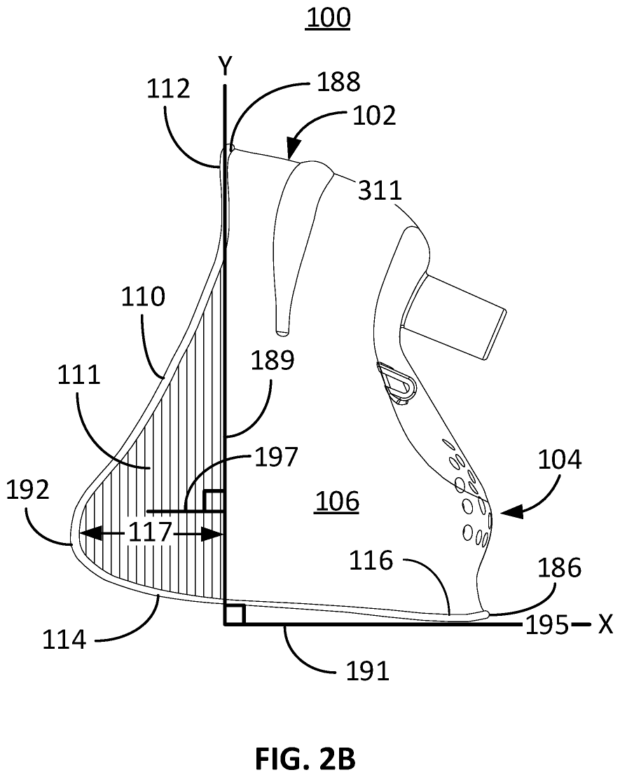

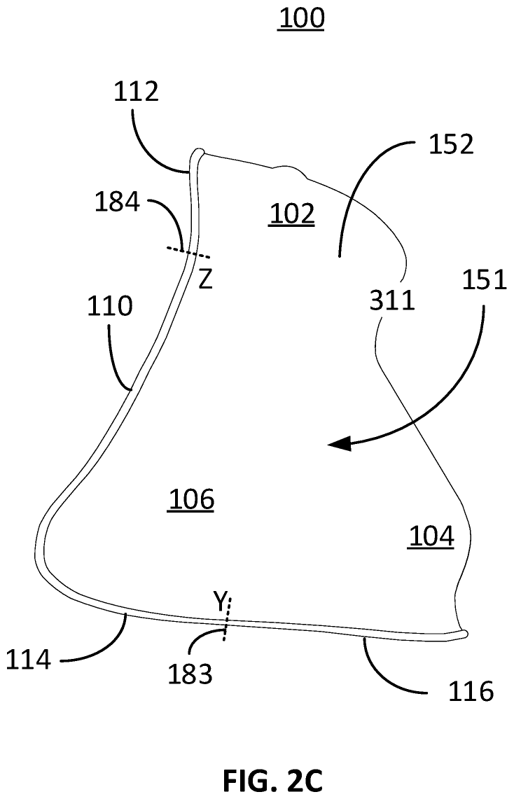

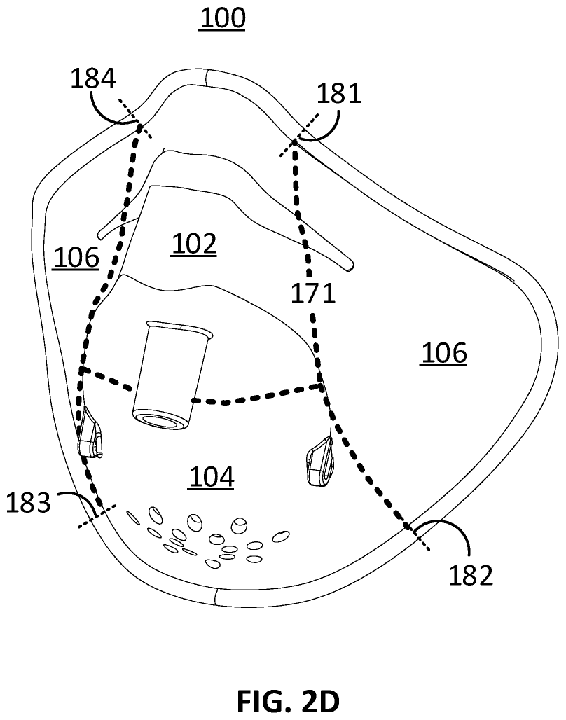

The facemask embodiment 100 is a clear polymer pliable cup-shaped facemask 100 that is essentially defined by a nose-covering region 102, a mouth-covering region 104, and two cheek-covering regions 106 within a facemask perimeter 110. In this embodiment, the nose-covering region 102 is shaped to closely conform to the person's nose 140, the mouth-covering region 104 is in front of the person's mouth 142, and the cheek-covering regions 106 is at least partially in contact with the person's cheek 144. The mouth-covering region 104 comprises a plurality of perforations 105 (see FIG. 2A) to allow airflow into and out from the person's mouth 142. The facemask 100 fits closely over the person's chin 148 and over the bridge of their nose 140. In the present embodiment, the facemask 100 is secured tightly to the person's face 200 via an elastic strap 150 that elastically compresses the facemask 100 closely to or against the person's face 200 between their nose 140 and mouth 142.

FIG. 2A is an isometric line drawing of the facemask embodiment 100 consistent with embodiments of the present invention. In more detail, the facemask perimeter 110 is a contiguous outer rim along the facemask cup 151 (see FIG. 2C) that is essentially in contact with the bridge 141 of a person's nose 140, a person's cheeks 144 and a person's chin 148. For purposes of explanation, the facemask perimeter 110 is essentially defined by a nose bridge rim 112 that is between the dotted lines W 181 and Z 184, a first cheek rim 114A between dotted lines W 181 and X 182, a second cheek rim 114B that is between dotted lines Y 183 and Z 184, and a chin rim 116 that is between dotted lines X 182 and Y 183. There are two strap anchors 118 between the inlet tube 120 and the mouth region 104. The strap anchors 118 are configured and arranged to anchor a head strap 150 to the facemask 100 either by a looped-hole (as shown), or some other kind of attachment configuration understood by those skilled in the art. The two strap anchors 118 are on either side of a facemask midline 311, which in certain embodiments the anchors 118 are less than 1.25 inches on either side of the facemask midline 311. The facemask midline 311 (a line in the figure) is defined as the midline bisecting the facemask 100 along the center of the nose region 102 and the mouth region 104 wherein the two cheek regions 106 are on either side of the facemask midline 311 as shown.

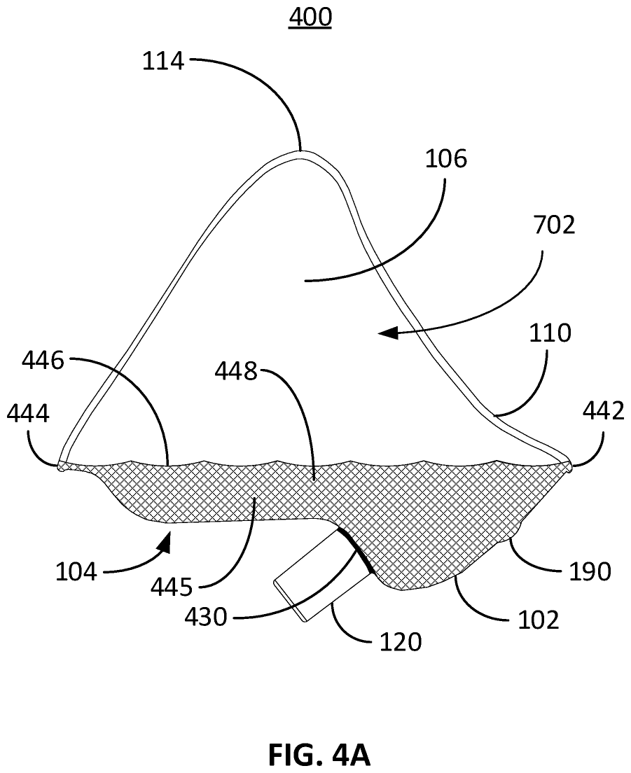

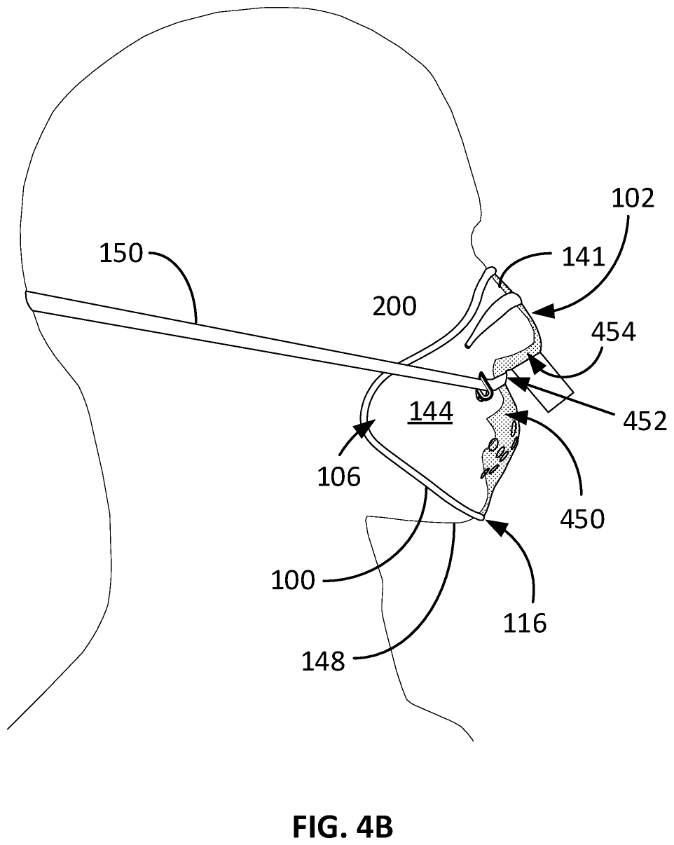

The facemask 100 further comprises a nose bridge reinforcement spring 190, a spring member to stiffen or otherwise retain the facemask to be shaped over the bridge 141 of the person's nose 140 (see FIG. 4B). The nose bridge reinforcement spring 190 can be a thickened portion of the clear molded facemask, a metal clip, or some other kind of spring-like member attached to the nose-region of the facemask 102. The cheek-covering regions 106 are flared 111 between one-half an inch and 2 inches longer 117 than a standard prior art facemask perimeter 119, illustratively depicted by the dashed line 119. The inlet tube 120 extends outwardly pointing downward at an angle of 48° when positioned on a neutral person's face 200. A passageway 122 that leads through a distal tube aperture 124 into the facemask 100 via a proximal tube aperture 126 (illustratively shown by the dashed lines) generally defines the inlet tube 120. Certain embodiments envision facemask 100 being a unitary molded clear PVC or some other polymer within the scope and spirit of the present invention.

FIG. 2B illustratively depicts a side view line drawing of a facemask embodiment showing one of two touch point flares consistent with embodiments of the present invention. The side view of the facemask 100 is referenced against a 90° reference angle 195 to better show the touch point flare 111 with respect to the rest of the facemask 100. The Y-axis 189 of the 90° angle 195 extends approximately through the nose bridge rim apex 188 on the facemask midline 311 and the X-axis 191 extends approximately through the chin rim apex 186, also along the facemask midline 311. The touch point flare 111 is depicted in the shaded region 111 and is defined as a flared portion of the cheek-covering region 106 that extends to the left of the Y-axis 189. As shown, a touch point flare 111 extends to the left side of the cheek-covering region 106 all the way to the cheek rim 114 with the flare length 117 defined in the X direction to the cheek rim apex 192. More specifically, each of the cheek rims 114 extend away from the mouth covering region 104 in the elongated flared lateral touch region 111 at least one inch at a right angle 197 from the vertical reference line 189 to the cheek rim apex 192. Other embodiments contemplate a flare length 117 of up to 2.5 inches long (to the cheek rim apex 192). The large flared sections 111 increase the touchpoint/contact percentage of the facemask 100 with a person's face 200 of 20% to 70% more than any prior art facemask, thereby increasing facemask seal, comfort and efficiency.

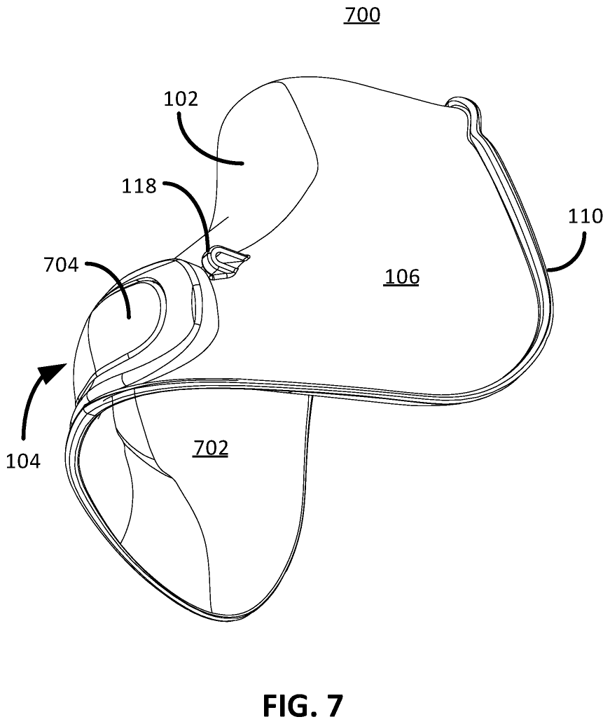

FIG. 2C is a side view line drawing of just the facemask cup portion of a facemask embodiment consistent with embodiments of the present invention. As shown, the facemask cup 151 is essentially defined herein by the nose-covering region 102, the mouth-covering region 104, two cheek-covering regions 106 within the perimeter 110. The inlet tube 120 and strap anchors 118 are not part of the facemask cup 151 for purposes of this description. The nose bridge rim 112, two cheek rims 114 and the chin rim 116 essentially define the perimeter 110. As previously discussed, the nose bridge rim 112 is between the dotted lines W 181 and Z 184, the cheek rim 114 is between the dotted lines Y 183 and Z 184, and the chin rim 116 is between dotted lines X 182 and Y 183. The outer surface 152 of the facemask cup 151 is viewably shown. Not viewable is the inside/inner surface 702 (see FIG. 7), which is the opposite side of the outer surface 152. The inside surface 702 is configured to either contact or face a person's face 200 albeit with some space between the inside surface 702 and the person's face 200. Certain embodiments envision at least 75% of the interior portion 702 of the facemask cup 151 being within ⅛ of an inch from a human face 200. Yet other embodiments envision at least 80% of the interior portion 702 of the facemask cup 151 being within ⅛ of an inch from a human face 200. While still other embodiments envision at least 90% of the interior portion 702 of the facemask cup 151 being within ⅛ of an inch from a human face 200. Yet other embodiments envision no part of the interior portion 702 of the facemask cup 151 being more than 0.35 inches from a human face 200.

FIG. 2D illustratively depicts the location of the different regions in a facemask embodiment consistent with embodiments of the present invention. The thick dotted lines 171 illustratively demarcate the different regions of the facemask embodiment 100. For example, the cheek-covering regions 106 are clearly segmented from the nose-covering region 102 and the mouth-covering region 104. It should be appreciated that the different regions 102, 104 and 106 do not necessarily have to follow the separation at the dotted lines 171 but rather can approximate the regions without departing from the scope and spirit of the present invention.

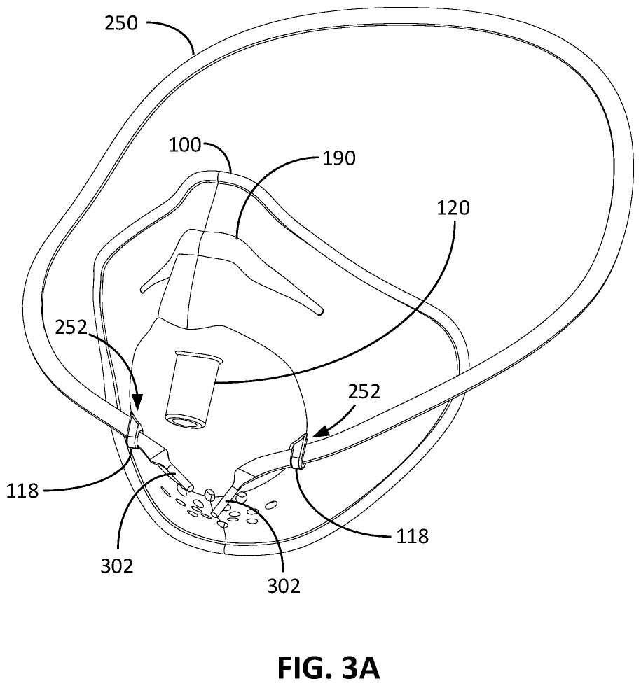

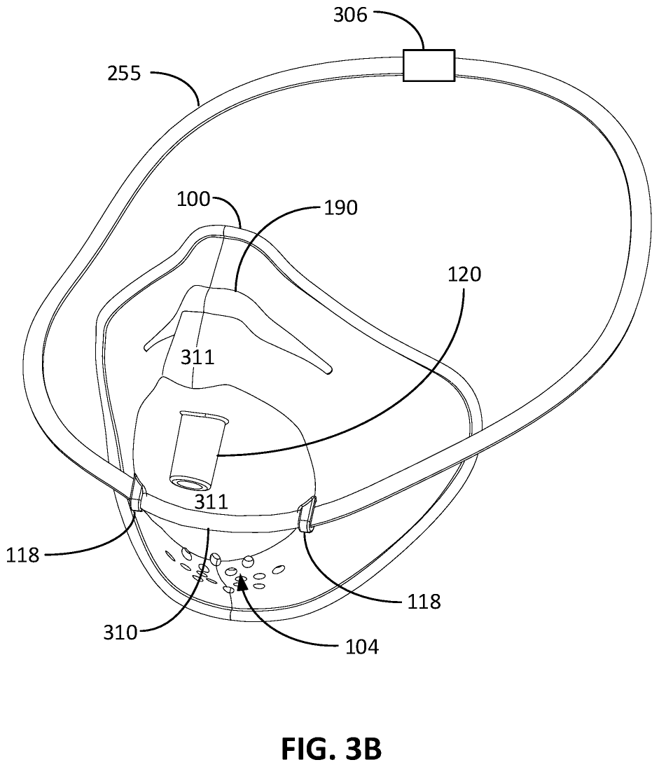

FIGS. 3A and 3B are isometric line drawings of a facemask embodiment with various head strap arrangements consistent with embodiments of the present invention. FIG. 3A shows the facemask 100 with a head strap 250 having two leads 302 that can be pulled or tightened by someone securing the facemask 100 to a person's face 200. The head strap 250 is threaded through the strap anchors 118 as shown. The head strap 250 can be elastic or non-elastic. Certain embodiments envision various anchoring arrangements other than the strap anchors 118, which can include simply stapling or affixing the head strap 250 at or near the anchor points 252. Certain other embodiments envision the head strap 250 being fixed to the head strap anchor locations 252 with the head strap 250 being elastic or being tightened from the back of a person's head 101.

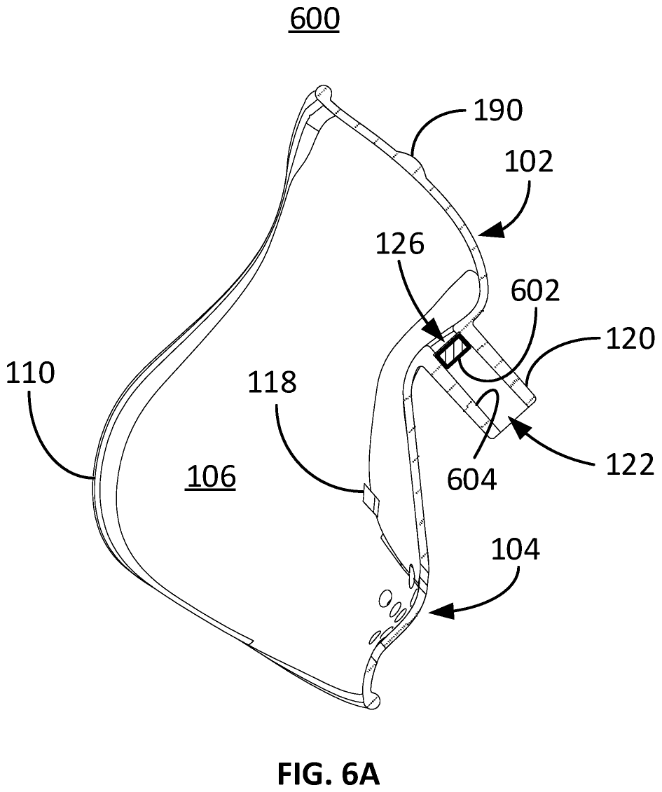

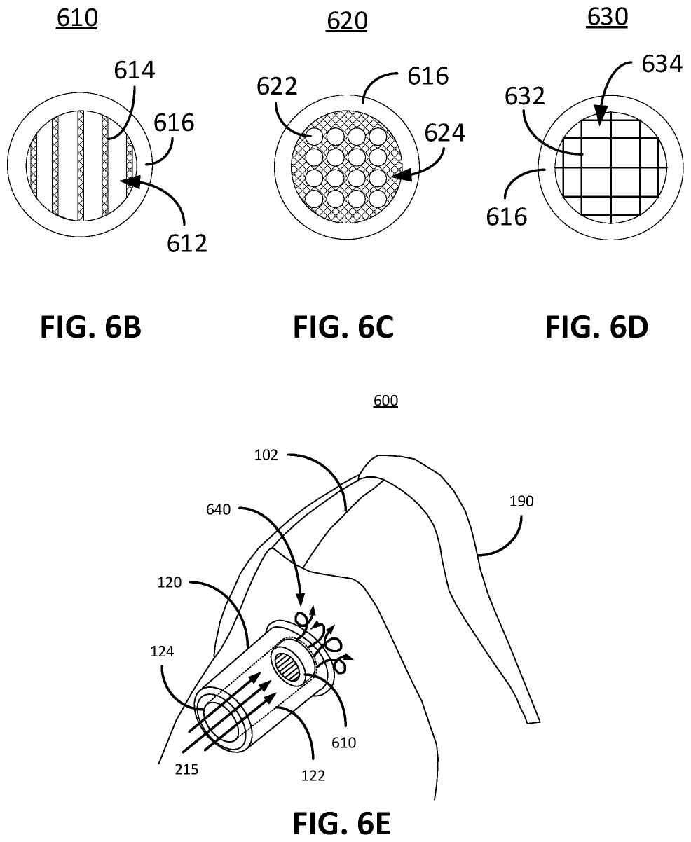

FIG. 3B shows the facemask 100 with an optional head strap 255 that essentially forms a continuous loop/band under the inlet tube 120 and above the mouth-region 104 (the FIG. 1). The head strap 255 is preferably elastic, however it should be appreciated that the head strap 255 is not limited to an elastic strap. The head strap 255 can be attached or tightened in the back at location 306 or optionally can be tightened in a different location. One benefit of the head strap 255 looping between the inlet tube 120 and the mouth-region 104 (i.e., crossing the midline 311) is that the strap portion 310 can pull the pliable facemask 100 towards the person's face 200 thereby reducing dead space 450 in the facemask 100 (see FIG. 6B). Dead space 450 is defined as any space between the interior region 702 of the facemask 400 and a person's face 200. Reduced dead space 450 allows the facemask 100 to be more efficient, requiring less pure oxygen to operate while supplying the oxygen at a higher concentration to the patient 101 than what any prior art facemask is capable of because there will be less CO₂ exhalent diluting the incoming O₂.

FIGS. 4A and 4B illustratively depict line drawings of interior volume and dead space of a facemask embodiment of an adult facemask consistent with embodiments of the present invention. FIG. 4A shows a facemask embodiment 400 tipped facing down in a position that holds essentially a maximum amount of water 445 assuming there are no perforations in the mouth region 104 and the proximal tube aperture 126 is sealed by a watertight plug 430. The facemask cup volume 448 is equivalent to the maximum amount of water 445 that a sealed facemask 400 can hold. As shown, the top of the water 446 essentially spans facemask 400 from the lowest chin rim point 444 to the lowest nose rim point 442 (i.e., any excess water 405 will simply spill over the rim 110) filling the interior cup volume 488. The lowest chin rim point 444 and the lowest nose rim point 442 likely correspond to the chin rim apex 186 and the nose bridge rim apex 188, respectively. The interior portion 702 of the facemask 400 is defined as the interior surface of the facemask 400 that interfaces a person's face 200, which does not include the inlet tube passageway 122. That is, the interior portion 702 either touches a person's face 200 or is otherwise directly opposed to the person's face 200 with some space 450 between the interior portion 702 of the facemask 400 and the person's face 200. The cup volume of a typical prior art adult facemask (not shown) is approximately 160 cc, or 9.8 in³, while certain embodiments envision the cup volume 448 of the present adult facemask 400 being less than 100 cc, or 6.1 in³. Other embodiments envision facemask 400 comprising a cup volume 448 less than 60 cc, or 3.7 in³. The lower the cup volume 448, the lower the dead space 450. Of course, there needs to be a certain amount of cup volume 448 for the person's face 200 to fit inside the facemask 400. An adult facemask is that which is used on essentially a fully grow man or woman's face, typically over the age of 12 years old. A child's facemask is smaller to fit a child's head so the measurements values disclosed will be skewed. Certain embodiments envision the values for an adult sized facemask being proportionally readjusted to correspond to a smaller sized child's facemask without departing from the scope and spirit of the present invention.

FIG. 4B illustratively depicts the dead space 450 of the facemask embodiment 400 when worn on a person's face 200 consistent with embodiments of the present invention. As shown, the facemask 100 conforms closely to the person's face 200 with certain embodiments contemplating less than 100 cc (6.1 in³) dead space 450 when worn on a patient's face 200. There is little dead space 450 between the bridge the person's nose 141 and the nose-covering region 102, however there is more dead space 450 between the person's nose 140 and the inlet tube 120 as shown by the dead space arrow 454. The head strap 150 is shown pulling the pliable facemask 100 against the person's face 200 under their nose 140 and above their mouth 142, across the midline of the facemask 400, to reduce dead space 450 in the front of their face 200. The head strap configuration 150 (either with the strap portion 310 or without the strap portion 310 so long as the head strap anchors 118 are within 1.25 inches from the midline 311, and preferably less than 1 inch from the midline 311) actively reduces dead space of the facemask cup 151 by pulling the facemask 400 into the person's face between their nose 140 and upper lip. The cheek-covering regions 106 and a significant portion of the nose-covering region 102 are pressed up against the person's face 200 while the mouth-covering region 104 has the majority of the dead space 450 to accommodate the natural movement of a person's mouth 142. Reduction in dead space 450 significantly improves the quality of oxygen in the facemask 100 provided by an oxygen source (such as an oxygen canister) from the inlet tube 120. Moreover, a reduction in dead space 450 reduces the amount of oxygen wasted by flowing out of the mouth region perforations 105 thereby increasing efficient use of oxygen provided by the oxygen source.

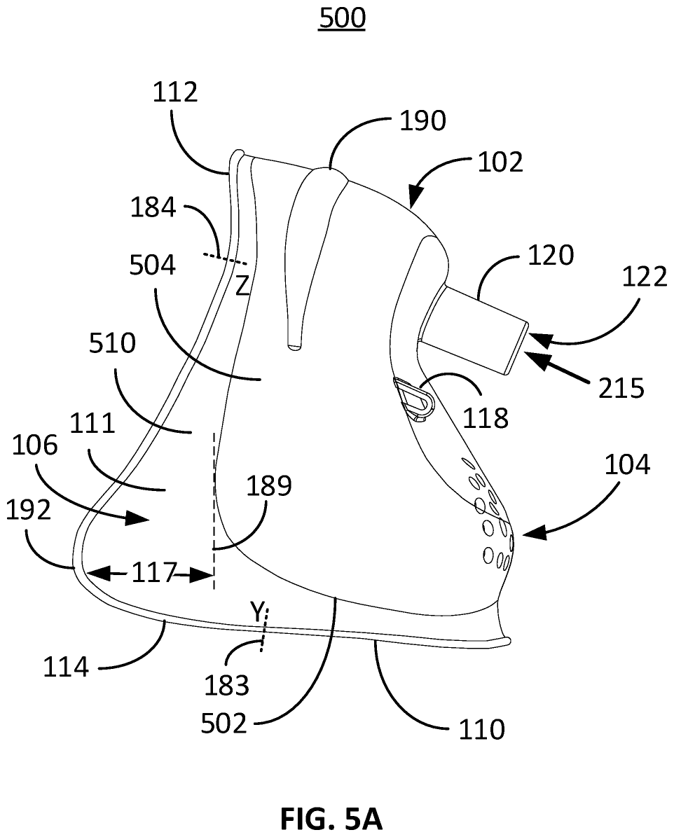

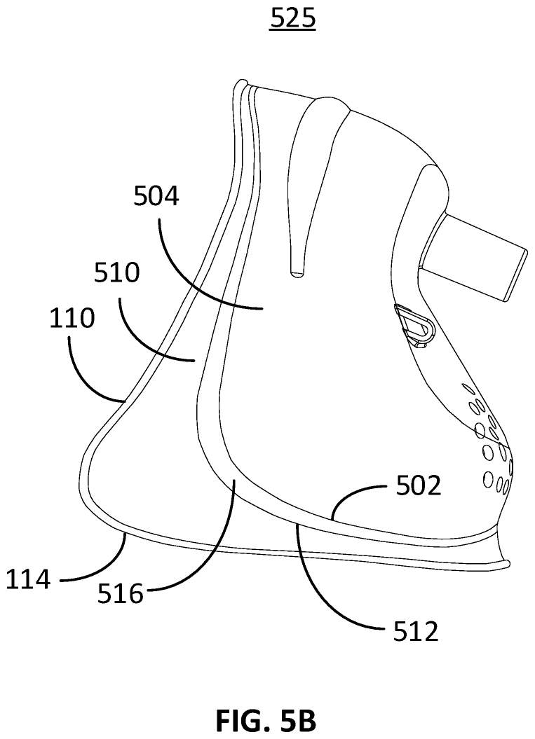

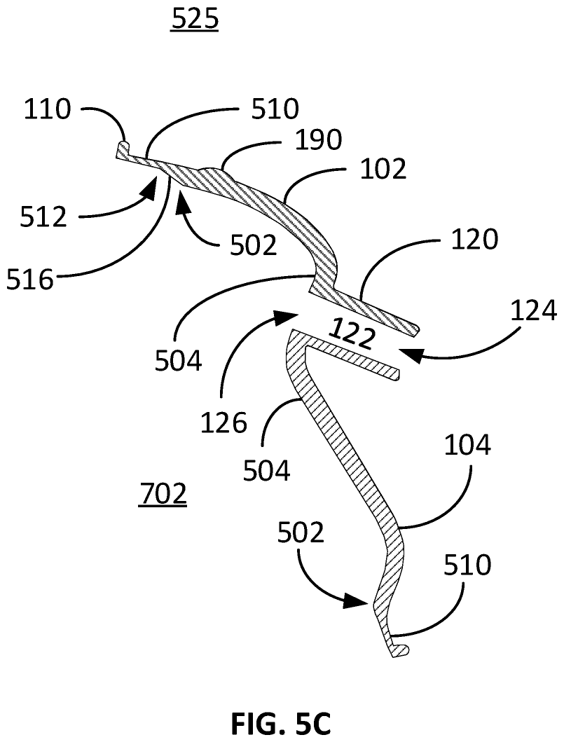

FIGS. 5A-5C are side view line drawings of a variable stiffness facemask embodiment consistent with embodiments of the present invention. FIGS. 5A-5C illustratively depict different structures and arrangements for accomplishing a low deflection region in a variable stiffness facemask. FIG. 5A shows the side view of a facemask embodiment 500 with a low stiffness region 510 and a high stiffness region 504. Certain embodiments contemplate the facemask 500, and more specifically the facemask cup 151, being a singular/common pliable material wherein the low stiffness region 510 is thinner than the high stiffness region 504. In some embodiments, the low stiffness region 510 is between 40% and 70% of the thickness of the high stiffness region 504. Other embodiments contemplate the low stiffness region 510 being as much as 90% of the thickness of the high stiffness region 504. Some embodiments envision a clear/semi-clear PVC (polyvinyl chloride) facemask with a high stiffness region 504 being approximately 0.06+/−0.01 inches thick and the low stiffness region 510 being 0.03+/−0.01 inches thick. Certain embodiments envision the perimeter 110 having a rim thickness of 0.09+/−0.01 inches thick and the nose bridge reinforcement spring 190 having a thickness of 0.155+/−0.2 inches thick. This particular facemask embodiment 500 possesses flared lateral touch region 111 that extends along each side of a person's cheek 144 to improve the seal of the facemask against a person's face 200. The flared lateral touch region 111 extends approximately from where the high stiffness region 504 interfaces 502 or otherwise meets the low stiffness region 510 to an apex 192 at the cheek rim 114. The high to low stiffness interface 502 does not necessarily equal the shape of a prior art facemask, but rather is used here to provide a sense of the shape of the flared lateral touch region 111. The flared region 111 is essentially between dotted lines Y 183 and Z 184 and covers a portion of the cheek-covering region 106. For reference, an arrow showing the direction of laminar inlet airflow 215 is provided from an air source (not shown), such as an oxygen tank or other oxygen source into the passageway 122 of the inlet tube 120. Certain embodiments envision the nose-covering region 102 being thicker than the average high stiffness region 504. Yet, other embodiments envision an upper lip region between the inlet tube 120 and the mouth region perforations 105 being thinner than the average high stiffness region 504 to facilitate easier deflection of the facemask 500 towards a human face 200 to reduce dead space 450.

Some other embodiments envision the facemask 500 not necessarily having different thickness material, but rather different kinds of materials. More specifically, the low stiffness region 510 could be composed of a different more flexible/lower stiffness material than the high stiffness region 504. Hence, better conformity and comfort can be accomplished with the facemask 500 being comprised of at least two different materials, one less stiff material in the low stiffness region 510 and a different stiffer material in the high stiffness region 504. The stiffer areas (and in some embodiments, the thicker areas) provide the needed integrity to maintain the facemask's shape so as not to compromise a wearer's breathing. The more pliable areas (thinner areas in some embodiments) provide greater dynamic deformity facilitating more comfort and a better seal of the facemask 500 against a person's cheek 144 than prior art facemasks.

FIG. 5B is a side view drawing of a different facemask embodiment 525 consistent with embodiments of the present invention. In this embodiment, the facemask 525 possesses a more gradual transition zone 516 between the high stiffness region 504 and the low stiffness region 510. The transition zone 516 could also be more of a step between the high stiffness region 504 and the low stiffness region 510, defined between the high-to-medium interface 502 and the low-to-medium interface 512. Some examples can include the transition zone 516 having a middle thickness compared to the high stiffness region 504 and the low stiffness region 510. For example, if the facemask 525 is essentially composed of clear/semi-clear flexible PVC with a high stiffness region 504 being approximately 0.06+/−0.01 inches thick and the low stiffness region 510 being 0.03+/−0.01 inches thick, the transition zone 516 could be 0.045+/−0.01 inches thick on average. Optionally, the facemask 525 could be composed of at least two different materials with the transition zone 516 being a blend of the materials used for the high stiffness region 504 and the low stiffness region 510. Yet another option contemplates the facemask 525 being composed of at least three different materials with low stiffness region 504 being a low stiffness material, the transition zone 516 being a medium thickness material, and the high stiffness region 504 being a high stiffness material. In this embodiment, the three different materials can be the same thickness or different thicknesses.

FIG. 5C illustratively shows a cross-section line drawing of the facemask embodiment of FIG. 5B consistent with embodiments of the present invention. A cross-section of 525 is shown by the crosshatched area taken along the profile of FIG. 5B. The perimeter 110 is a band of thicker material than the low stiffness region 510. The transition zone 516 tapers from the low-to-medium interface 512 at the low stiffness region 510 to the high-to-medium interface 502 in the high stiffness region 504. The nose bridge reinforcement spring 190 in the nose-covering region 102 is the thickest portion of the facemask 525. From this cross-sectional perspective, the inlet tube 120 clearly shows the distal tube aperture 124 and the proximal tube aperture 126 that form an open pathway into the inlet tube passageway 122. In this particular embodiment, the mouth-covering region 104 is approximately the same thickness as the rest of the high stiffness region 504.

FIGS. 6A-6E illustratively depict various diffuser embodiments located in the inlet tube passageway of a facemask embodiment consistent with embodiments of the present invention. FIG. 6A illustratively shows a cross-section profile view and outline of the facemask embodiment 600 with a diffuser 602 disposed in the inlet tube passageway 122. The facemask embodiment 600 comprises many common elements of the other facemask embodiments, such as the perimeter rim 110, a nose bridge reinforcement spring 190 in the nose-covering region 102, a strap anchor 118 above the mouth-covering region 104 and a cheek-covering region 106. In the present embodiment, the diffuser 602 is disposed near or at the proximal tube aperture 126 to cause disruption of laminar inlet airflow 215.

FIGS. 6B-6D illustratively show various diffuser embodiments that can be placed in the inlet tube passageway 122 consistent with embodiments of the present invention. FIG. 6B shows a diffuser embodiment 610 comprising a plurality of diffuser slots 614 framed by a diffuser cuff 616. Forced air (pressurized air) from an oxygen source can be made to flow through the slotted diffuser space 612 between the diffuser slots 614 to disrupt the laminar flow coming in the tube inlet aperture 124. FIG. 6C shows a diffuser embodiment 620 comprising a plurality of circular diffuser holes 622 that perforate a diffuser barrier 624 framed by a diffuser cuff 616. Forced air from the oxygen source can be made to flow through the circular diffuser holes 622 into the facemask 600 to disrupt the incoming laminar flow. FIG. 6D shows yet another diffuser embodiment 630 comprising a screen 632 framed by a diffuser cuff 616. Forced air from the oxygen source can be made to flow through the space 634 between the wires/members 632 that make up the screen 632 to disrupt the incoming laminar flow. As should be appreciated, the diffuser cuffs 616 can be affixed (e.g., adhered, bolted, glued, etc.) to the interior inlet tube wall 604 of the nose inlet tube 120. The various diffuser embodiments 610, 620 and 630 are merely example species of a generic diffuser concept wherein there are a number of different conceivable diffuser embodiments consistent with the scope and spirit of the present invention.

FIG. 6E illustratively depicts disruptive flow from laminar intake flow through a diffuser embodiment consistent with embodiments of the present invention. Here, a slotted diffuser 610 is attached to the interior inlet to wall 604 approximately at the proximal tube aperture 126 of the facemask 600. The nose-covering region 102 and the nose bridge reinforcement spring 190 are shown for reference. An oxygen tube (not shown) is attached to the nose inlet tube 120 through which laminar inlet airflow 215 is forced into the tube inlet aperture 124 through the inlet tube passageway 122 and disrupted into turbulent inlet flow 640 when the laminar inlet airflow 215 passes through the diffuser slots 614. In this way, the turbulent inlet flow 640 is less harsh on a wearer's nostrils 206.

FIG. 7 is a line drawing of yet a different embodiment of a filter facemask without an air inlet port and a filter over the mouth region consistent with embodiments of the present invention. Because filter facemask embodiment 700 depicts a protective facemask rather than a rebreather facemask, there is no air inlet port in the nose-covering region 102 and a filter (such as an N-95 filter, for example) over the one or more perforations in the mouth-covering 104. The interior cup region 702 can benefit from many of the attributes of the facemask embodiments previously discussed including a flared lateral touch region 111 and placement of the strap anchors 118.

With the present description in mind, below is a summary of some embodiments consistent with the present invention. The elements called out below are examples provided to assist in the understanding of the present invention and should not be considered limiting.

One embodiment of the present invention contemplates a variable stiffness facemask 100 comprising: a facemask cup 151 that is essentially defined by a nose covering region 102, a mouth covering region 104, two cheek coving regions 106 and a perimeter 110. A nose bridge rim 112, two cheek rims 114 and a chin rim 116 essentially define the perimeter 110. The nose covered region 102 is shaped to closely conform to a human nose 140, the mouth covering region 104 configured to be in front of a human mouth 142, and the cheek covering regions 106 configured to at least partially contact a corresponding sides of a human cheek 144. The term “shaped to” should be construed to mean “configured to conform by way of shape” within the scope and spirit of the present invention. An inlet tube 120 outwardly extends from the facemask 100, the inlet tube 120 generally defined by a passageway 122 that leads through a distal aperture 124 and through a nose aperture 126 in the facemask 100. At least one strap anchor 118 is between the inlet tube 120 and the mouth covering region 104. And, a low force deflection band 130 that extends at least 0.3 inches from the perimeter 110 towards the mouth covering region 104, the low force deflection band 130 is at least 15% more pliable than the mouth covering region 104.

The variable stiffness facemask 100 embodiment further envisioning wherein the low force deflection band 130 is at least 10% thinner than the rest of the facemask 100.

The variable stiffness facemask 100 embodiment further envisioning wherein each of the cheek rims 114 interface with the chin rim 116 in a flared lateral touch region 111. This embodiment further envisions wherein the flared lateral touch region 111 extends from one of the cheek covering regions 106 between 0.5 inch and 1.5 inch. Optionally this embodiment further envisions wherein the flared lateral touch region 111 is part of the low force deflection band 130.

The variable stiffness facemask 100 embodiment further contemplating wherein the low deflection band 130 comprises a more pliable material than the rest of the facemask 100.

The variable stiffness facemask 100 embodiment further considering wherein the facemask 100 is a unitary polymer structure.

The variable stiffness facemask embodiment further envisioning wherein the low deflection band 130 is defined by variable thicknesses that provide variable stiffness.

The variable stiffness facemask 100 embodiment further imagining wherein the nose bridge rim 112, the two cheek rims 114 and the chin rim 116 are thicker than the low deflection band 130.

The variable stiffness facemask 100 embodiment further comprising perforations through the mouth covering region 104.

The variable stiffness facemask 100 further considering wherein at least part of the nose covering region 102 is thicker than the rest of the facemask 100.

The variable stiffness facemask 100 embodiment further contemplating wherein the inlet tube 120 extends from the facemask 100 at between 30° and 60° when worn on a human face.

The variable stiffness facemask 100 embodiment further imagining wherein the at least one strap anchor 118 is within 1.25 inches from a facemask midline, which is defined as extending along the center of the facemask 100 bisecting the nose covering region 102 and the mouth covering region 104.

Another embodiment includes a nasal directed flow facemask 100 comprising: a pliable facemask cup 151 that is essentially defined by a nose covering region 102, a mouth covering region 104, two cheek coving regions 106 and a perimeter 110, the perimeter 110 essentially defined by a nose bridge rim 112, two cheek rims 114 and a chin rim 116. The nose covered region 102 is shaped to closely conform to a human nose 140, the mouth covering region 104 is configured to be in front of a human mouth 142, the cheek covering regions 106 are configured to at least partially contact a corresponding sides of a human cheek 144. An inlet tube 120 outwardly extends from the facemask 100, the inlet tube 120 is generally defined by a passageway 122 that leads through a distal aperture 124 and a proximal nose aperture 126 in the facemask 100. The inlet tube 120 extends in a downward direction from the facemask 100 at an angle 160 between 30° and 60° from horizontal 162 when the facemask 100 is worn on a human face 200 that is in a neutral position 205.

The nasal directed flow facemask 100 embodiment further comprising at least one strap anchor 118 between the inlet tube 120 and the mouth covering region 104 configured to retain a head retention strap 150, the at least one strap anchor 118 is within 1.25 inches from a facemask midline 311, the facemask midline 311 is defined as extending along the center of the facemask 100 bisecting the nose covering region 102 and the mouth covering region 104.

The nasal directed flow facemask 100 embodiment further contemplating wherein the angle 160 is between 42° and 50°.

The nasal directed flow facemask 100 embodiment further envisioning wherein the nose aperture 126 is configured to direct airflow 215 into a human's nostrils 206 at approximately at the angle 160 which is approximately a nostril angle of the nostril 206.

The nasal directed flow facemask 100 embodiment further comprising a diffuser 210 that disrupts laminar flow 216 of airflow 215. This embodiment further contemplating wherein the diffuser 210 is selected from a group comprising obstructing slots 222, obstructing grids 224, and a plurality of perforations 105.

The nasal directed flow facemask 100 embodiment further comprising perforations through the mouth covering region 104.

The nasal directed flow facemask 100 embodiment further imagining wherein the facemask 100 is a unitary polymer structure.

The nasal directed flow facemask 100 embodiment further considering wherein the inlet tube 120 and the facemask 100 is a unitary polymer structure.

The nasal directed flow facemask 100 embodiment further contemplating wherein each of the cheek rims 114 extend away from the mouth covering region 104 in an elongated flared lateral touch region 111 that is at least one inch in length 117 to a cheek rim apex 192 at a right angle 197 from a vertical reference line 189 defined by a 90° reference angle 195 that passes through a face mask nose bridge rim apex 188 along the Y-axis 189 and the chin rim apex 186 along the X-axis 191.

The nasal directed flow facemask 100 embodiment further imagining wherein the inlet tube 120 is approximately in-line with the nose covering region 102 along a facemask midline 311 that is defined as extending along the center of the facemask 100 bisecting the nose covering region 102 and the mouth covering region 104.

While other embodiments include a reduced dead space adult facemask 100 comprising: a pliable adult sized facemask cup 151 that is essentially defined by a nose covering region 102, a mouth covering region 104, two cheek coving regions 106 and a perimeter 110, the perimeter 110 essentially defined by a nose bridge rim 112, two cheek rims 114 and a chin rim 116. The nose covered region 102 is shaped to closely conform to an adult human nose 140, the mouth covering region 104 is configured to be in front of an adult human mouth 142, the cheek covering regions 106 are configured to at least partially contact a corresponding sides of adult human cheeks 144. An inlet tube 120 outwardly extends from the adult facemask 100, the inlet tube 120 is generally defined by a passageway 122 that leads through a distal aperture 124 and nose aperture 126 in the facemask 100. A facemask cup volume 302 is essentially defined by a maximum amount of water the facemask cup 151 can hold. The facemask cup volume 302 is less than 6.1 in³.

The reduced dead space adult facemask 100 embodiment further comprising at least one strap anchor 118 between the inlet tube 120 and the mouth covering region 104 configured to retain a head retention strap 150, the strap anchor 118 is less than 1.25 inches from a facemask midline 311 defined as extending along the center of the facemask 100 bisecting the nose covering region 102 and the mouth covering region 104.

The reduced dead space adult facemask 100 embodiment further contemplating wherein no part of an interior portion 702 of the facemask cup 151 is more than 0.35 inch from a human face 200, the interior portion 702 is defined as the interior surface of the facemask cup 151 that is configured to face the person's face 200.

The reduced dead space adult facemask 100 embodiment further considering wherein at least 90% of an interior portion 702 of the facemask cup 151 is within one eighth of an inch from a human face 200, the interior portion 702 is defined as the interior surface of the facemask cup 151 that is configured to face the person's face 200.

The reduced dead space adult facemask 100 embodiment further comprising a head strap 150 that crosses over a facemask midline 311 between the inlet tube 120 and the mouth covering region 104, the facemask midline 311 is defined as extending along the center of the facemask 100 bisecting the nose covering region 102 and the mouth covering region 104. This embodiment further contemplating wherein the head strap 150 possesses a strap portion 310 that spans between two head strap anchors 118 that are between the inlet tube 120 and the mouth covering region 104, the head strap anchors 118 are less than 1.25 inches from the facemask midline 311, the strap portion 310 is configured to deflect the facemask 100 towards a person's face 200 when facemask 100 is worn by a person 101. This can further be wherein the head strap 150 is a contiguous elastic band.

The reduced dead space adult facemask 100 embodiment further envisioning wherein the facemask 100 is a unitary polymer element.

The reduced dead space adult facemask 100 embodiment further cimagining wherein each of the cheek rims 114 extend away from the mouth covering region 104 in an elongated flared lateral touch region 111 at least one inch in length 117 to a cheek rim apex 192 at a right angle 197 from a vertical reference line 189 defined by a 90° reference angle 195 that passes through the face mask nose bridge rim apex 188 along the Y-axis 189 and the chin rim apex 186 along the X-axis 191.

Yet another embodiment of the present invention contemplates a pliable adult facemask 100 comprising: a facemask cup 151 that is essentially defined by a nose covering region 102, a mouth covering region 104, two cheek coving regions 106 and a perimeter 110, the perimeter 110 essentially defined by a nose bridge rim 112, two cheek rims 114 and a chin rim 116. The nose covered region 102 being shaped to closely conform to an adult human nose 140, the mouth covering region 104 being configured to be in front of an adult human mouth 142, the cheek covering regions 106 being configured to at least partially contact a corresponding sides of an adult human cheek 144. An inlet tube 120 can outwardly extend from the adult facemask 100. A passageway 122 that leads through distal aperture 124 and nose aperture 126 in the facemask 100 generally defines the inlet tube 120. At least 80% of an interior portion 702 of the facemask cup 151 is within one eighth of an inch from a human face 200.

The pliable adult facemask 100 embodiment further comprising at least one strap anchor 118 between the inlet tube 120 and the mouth covering region 104 configured to retain a head retention strap 150, the strap anchor 118 is less than 1.25 inches from a facemask midline 311 defined as extending along the center of the facemask 100 bisecting the nose covering region 102 and the mouth covering region 104.

The pliable adult facemask 100 embodiment further contemplating wherein no part of an interior portion 702 of the facemask cup 151 is more than 0.35 inch from a human face 200.

The pliable adult facemask 100 embodiment further imagining wherein at least 90% of an interior portion 702 of the facemask 100 is within one eighth of an inch from a human face 200.

The pliable adult facemask 100 embodiment further comprising a head strap 150 that crosses over a facemask midline 311 between the inlet tube 120 and the mouth covering region 104, the facemask midline 311 is defined as extending along the center of the facemask 100 bisecting the nose covering region 102 and the mouth covering region 104. This can further include wherein the head strap 150 possesses a strap portion 310 that spans between two head strap anchors 118 that are between the inlet tube 120 and the mouth covering region 104, the head strap anchors 118 are less than 1.25 inches from the facemask midline 311, the strap portion 310 is configured to deflect the facemask 100 towards a person's face 200 when facemask 100 is worn by a person 101.

The pliable adult facemask 100 embodiment further envisioning wherein each of the cheek rims 114 extend away from the mouth covering region 104 in an elongated flared lateral touch region 111 at least one inch in length 117 to a cheek rim apex 192 at a right angle 197 from a vertical reference line 189 defined by a 90° reference angle 195 that passes through a face mask nose bridge rim apex 188 along the Y-axis 189 and the chin rim apex 186 along the X-axis 191.

The pliable adult facemask 100 embodiment further considering wherein a facemask cup volume 302 that is essentially defined by a maximum amount of water the facemask 100 can hold it is less than 5 in³.

The above embodiments are not intended to be limiting to the scope of the invention whatsoever because many more embodiments are easily conceived within the teachings and scope of the instant specification. Moreover, the corresponding elements in the above example should not be considered limiting.

It is to be understood that even though numerous characteristics and advantages of various embodiments of the present invention have been set forth in the foregoing description, together with the details of the structure and function of various embodiments of the invention, this disclosure is illustrative only, and changes may be made in detail, especially in matters of structure and arrangement of parts within the principles of the present invention to the full extent indicated by the broad general meaning of the terms in which the appended embodiments are expressed. For example, though the embodiments generally describe at least one mouth perforation 105 in a mouth-covering region 104, in any of the facemask embodiments, a filter, such as a HEPA filter, can be used over the mouth-covering region 104 covering plurality of mouth region perforations 105 to protect against contaminants including viruses and bacteria. It should be understood and appreciated that any element described in one embodiment can be equally used and/or substituted in place of a like element in other embodiments without departing from the scope and spirit of the present invention. Further, the terms “one” is synonymous with “a”, which may be a first of a plurality.

It will be clear that the present invention is well adapted to attain the ends and advantages mentioned as well as those inherent therein. While presently preferred embodiments have been described for purposes of this disclosure, numerous changes may be made which readily suggest themselves to those skilled in the art and which are encompassed in the spirit of the invention disclosed and as defined in the appended claims.

What is claimed is:

1. A variable stiffness facemask comprising:
a facemask cup that is essentially defined by a nose covering region, a mouth covering region, two cheek coving regions and a perimeter, the perimeter essentially defined by a nose bridge rim, two cheek rims and a chin rim,
the nose covered region shaped to closely conform to a human nose, the mouth covering region configured to be in front of a human mouth, the cheek covering regions configured to at least partially contact a corresponding sides of a human cheek;
an inlet tube outwardly extending from the variable thickness facemask, the inlet tube generally defined by a passageway that leads through a distal aperture and a nose aperture in the facemask;
at least one strap anchor between the inlet tube and the mouth covering region; and
a low force deflection band that extends at least 0.3 inches from the perimeter towards the mouth covering region, the low force deflection band is at least 15% more pliable than the mouth covering region.

2. The variable stiffness facemask of claim 1 wherein the low force deflection band is at least 10% thinner than the rest of the facemask.

3. The variable stiffness facemask of claim 1 wherein each of the cheek rims interface with the chin rim in a flared lateral touch region.

4. The variable stiffness facemask of claim 3 wherein the flared lateral touch region extends from one of the cheek covering regions between 0.5 inch and 1.5 inch.

5. The variable stiffness facemask of claim 3 wherein the flared lateral touch region is part of the low force deflection band.

6. The variable stiffness facemask of claim 1 wherein the low deflection band comprises a more pliable material than the rest of the facemask.

7. The variable stiffness facemask of claim 1 wherein the facemask is a unitary polymer structure.

8. The variable stiffness facemask of claim 1 wherein the at least one strap anchor is within 1.25 inches from a facemask midline, which is defined as extending along the center of the facemask bisecting the nose covering region and the mouth covering region.

9. A nasal directed flow facemask comprising:
a pliable facemask cup that is essentially defined by a nose covering region, a mouth covering region, two cheek coving regions and a perimeter, the perimeter essentially defined by a nose bridge rim, two cheek rims and a chin rim,
the nose covered region shaped to closely conform to a human nose, the mouth covering region configured to be in front of a human mouth, the cheek covering regions configured to at least partially contact a corresponding sides of a human cheek;
an inlet tube outwardly extending from the facemask, the inlet tube generally defined by a passageway that leads through a distal aperture and a proximal nose aperture in the facemask,
the inlet tube extending in a downward direction from the facemask at an angle between 30° and 60° from horizontal when the facemask is worn on a human face that is in a neutral position.

10. The nasal directed flow facemask of claim 9 further comprising at least one strap anchor between the inlet tube and the mouth covering region configured to retain a head retention strap, the at least one strap anchor is within 1.25 inches from a facemask midline, the facemask midline is defined as extending along the center of the facemask bisecting the nose covering region and the mouth covering region.

11. The nasal directed flow facemask of claim 9 wherein the angle is between 42° and 50°.

12. The nasal directed flow facemask of claim 9 wherein the nose aperture is configured to direct airflow into a human's nostrils at approximately at the angle which is approximately a nostril angle of the nostril.

13. The nasal directed flow facemask of claim 9 further comprising a diffuser that disrupts laminar flow of airflow.

14. The nasal directed flow facemask of claim 1 wherein each of the cheek rims extend away from the mouth covering region in an elongated flared lateral touch region that is at least one inch in length to a cheek rim apex at a right angle from a vertical reference line defined by a 90° reference angle that passes through a face mask nose bridge rim apex along the Y-axis and the chin rim apex along the X-axis.

15. A reduced dead space adult facemask comprising:
a pliable adult sized facemask cup that is essentially defined by a nose covering region, a mouth covering region, two cheek coving regions and a perimeter, the perimeter essentially defined by a nose bridge rim, two cheek rims and a chin rim,
the nose covered region shaped to closely conform to an adult human nose, the mouth covering region configured to be in front of an adult human mouth, the cheek covering regions configured to at least partially contact a corresponding sides of adult human cheeks;
an inlet tube outwardly extending from the adult facemask, the inlet tube generally defined by a passageway that leads through a distal aperture and nose aperture in the facemask,
a facemask cup volume that is essentially defined by a maximum amount of water the facemask cup can hold, the facemask cup volume less than 6.1 in³.

16. The reduced dead space adult facemask of claim 15 further comprising at least one strap anchor between the inlet tube and the mouth covering region configured to retain a head retention strap, the strap anchor is less than 1.25 inches from a facemask midline defined as extending along the center of the facemask bisecting the nose covering region and the mouth covering region.

17. The reduced dead space adult facemask of claim 15 wherein no part of an interior portion of the facemask cup is more than 0.35 inch from a human face, the interior portion is defined as the interior surface of the facemask cup that is configured to face the person's face.

18. The reduced dead space adult facemask of claim 15 wherein at least 90% of an interior portion of the facemask cup is within one eighth of an inch from a human face, the interior portion is defined as the interior surface of the facemask cup that is configured to face the person's face.

19. The reduced dead space adult facemask of claim 15 further comprising a head strap that crosses over a facemask midline between the inlet tube and the mouth covering region, the facemask midline is defined as extending along the center of the facemask bisecting the nose covering region and the mouth covering region.

20. The reduced dead space adult facemask of claim 19 wherein the head strap possesses a strap portion that spans between two head strap anchors that are between the inlet tube and the mouth covering region, the head strap anchors are less than 1.25 inches from the facemask midline, the strap portion is configured to deflect the facemask towards a person's face when facemask is worn by a person.
